(19) United States
(12) Patent Application Publication
(10) Pub. No.: US 2012/0116538 A1
(43) Pub. Date: May 10, 2012

(54) PROCESS TO ENHANCE FITTINGS INCLUDING NOVEL FLEXURE IMPARTED AND CUSTOMIZABLE LINERS AND SUSPENSION SLEEVES

(76) Inventor: W. Stanley Patterson, Irvine, CA (US)

(21) Appl. No.: 13/097,593

(22) Filed: Apr. 29, 2011

Related U.S. Application Data
(60) Provisional application No. 61/410,262, filed on Nov. 4, 2010.

(51) Int. Cl. A61F 2/78

(52) U.S. Cl. 623/36

(57) ABSTRACT

An improved liner or suspension sleeve having an upper zone aligned above an articulating joint of a body; a lower zone aligned below said articulating joint; and a medial zone of flexure comprising ridges aligned with an posterior portion of said articulating joint; whereby the region corresponding to an articulating joint or residuum has at least one of a different durometer and altered surface characteristics. The ridges allow flexion of the liner or suspension sleeve without substantial compression leading to buckling of the liner or suspension sleeve in the vicinity of the articulating joint.

The instant filing requesting grant of Letters Patent claims full Paris Convention Priority from U.S. Provisional Patent Application Ser. No. 61/410,262, filed Nov. 4, 2010 in the name of the present inventor, and which is expressly incorporated herein by reference as if fully set forth herein.

### BACKGROUND OF THE INVENTION

### Field of the Disclosure

The present disclosure relates to processes for making fitting of prosthetics better for amputees. In particular the instant disclosure teaches how inner-sleeves, prosthetic suspension sleeves, and/or other devices functioning as liners can be enhanced by imparting mechanically induced flexure at predetermined locations to avoid bunching, pinching and deleterious foldings. Such foldings often occur at articulating points, such as the popliteal fossa and the cubital fossa, for example, with conventional liner systems.

While there has been a confluence of activity around the improvement of prosthetic ankles, knees and especially feet, little or no attention has been devoted to amelioration of the interface between an amputee's residuum and any prosthetic appended thereto.

### SUMMARY

Briefly stated, an improved liner or suspension sleeve having an upper zone aligned above an articulating joint of a body; a lower zone aligned below said articulating joint; and a medial zone of flexure comprising ridges aligned with an posterior portion of said articulating joint; whereby the region corresponding to an articulating joint or residuum has at least one of a different durometer and altered surface characteristics. The ridges allow flexion of the liner or suspension sleeve without substantial compression leading to buckling of the liner or suspension sleeve in the vicinity of the articulating joint.

According to embodiments, a process for improving fitting of a liner, comprising, measuring a patient's residuum, generating a molded liner, and creating a zone of flexure in at least a portion of the molded liner.

According to embodiments, a liner, comprising, in combination, an upper zone aligned above an articulating joint of a body, a lower zone aligned below said articulating joint, and a medial zone comprising ridges aligned with a posterior portion of said articulating joint.

According to embodiments, a liner, comprising, in combination, a material of substantially homogenous thickness being tapered from an upper end to a lower end thereof, ridges in a region corresponding to an articulating joint or a residuum, the ridges having troughs of thickness lower than that of surrounding portions of the liner; whereby the region corresponding to an articulating joint or residuum has at least one of a different durometer and altered surface characteristics.

According to embodiments, a prosthetic suspension sleeve, comprising, in combination, a tubular shape, an upper zone of the tubular shape aligned above an articulating joint of a body, a lower zone of the tubular shape aligned below said articulating joint, and a medial zone of the tubular shape comprising ridges aligned with an posterior portion of said articulating joint.

According to embodiments, an enhanced liner system, which comprises, in combination, at least a layer of material designed to buffer the interface with a prosthetic; and at least one indented position positioned upon a predetermined location of said liner, enabling flexure of said layer to avoid bunching, pinching, or other insult to a user.

Disclosed herein, according to embodiments, is a process for improving fitting of a liner, comprising: measuring a patient's residuum; generating a molded liner; and creating a zone of flexure in at least a portion of the molded liner.

Further disclosed herein, according to embodiments, is a liner, comprising, in combination: an upper zone aligned above an articulating joint of a body; a lower zone aligned below said articulating joint; a medial zone comprising ridges aligned with an posterior portion of said articulating joint.

According to embodiments, there is disclosed an enhanced liner system which comprising, in combination at least a layer of material designed to buffer the interface with a prosthetic; and, at least one indented position positioned upon a predetermined location of said liner, enabling flexure of said layer to avoid bunching, pinching or other insult to a user.

### DRAWINGS

The above-mentioned features and objects of the present disclosure will become more apparent with reference to the following description taken in conjunction with the accompanying drawings wherein like reference numerals denote like elements and in which:

FIG. 1 is view of a liner, according to embodiments of the present disclosure;

FIG. 2 is view of a liner, according to embodiments of the present disclosure;

FIG. 3 is view of a liner, according to embodiments of the present disclosure;

FIG. 4 is view of a liner, according to embodiments of the present disclosure;

FIG. 5 is view of a liner, according to embodiments of the present disclosure;

FIG. 6 is view of a liner, according to embodiments of the present disclosure;

FIG. 7 is view of a liner, according to embodiments of the present disclosure;

FIG. 8 is view of a liner, according to embodiments of the present disclosure;

FIG. 9 is a cross-sectional view of a liner, according to embodiments of the present disclosure;

FIG. 10 is a cross-sectional view of a liner, according to embodiments of the present disclosure;

FIG. 11 is a cross-sectional view of a liner, according to embodiments of the present disclosure;

FIG. 12 is a cross-sectional view of a liner in an extended state with detailed view, according to embodiments of the present disclosure;

FIG. 13 is a cross-sectional view of a liner in a flexed state with detailed view, according to embodiments of the present disclosure;

FIG. 14 is view of a liner, according to embodiments of the present disclosure;

FIG. 15 is view of a liner, according to embodiments of the present disclosure;

FIG. 16 is view of a liner, according to embodiments of the present disclosure; and

FIG. 17 is view of a liner, according to embodiments of the present disclosure.

### DETAILED DESCRIPTION

The present inventors have discovered that it is possible to overcome bunching, particularly that behind the hamstring area of an amputee, by mechanically altering flexure of subject liners and prosthetic suspension sleeves. According to embodiments of the present disclosure, the teachings disclosed herein apply to liners, inner-sleeves, and prosthetic suspension sleeves. As such, references to anyone of these and other prosthetic products may be applied to any liner, inner-sleeve, prosthetic suspension sleeve, and other prosthetic products.

Among below the knee amputees, a longstanding problem is that the fabric cover and/or substrate materials of a liner bunches in the area by the hamstring of an amputee. This is extremely uncomfortable, and in fact interferes with the proper function and comfort of prosthetics.

An elegantly simple solution is offered for consideration herein, namely decreasing the extra material and fabric, creating mechanical relief and detenting, to accommodate fitting a liner or suspension sleeve by creating zones of flexure and troughs within the same.

Prosthetic liners are worn on residual limbs to cushion the contact area between the residual limb and a prosthetic socket. Prosthetic liners include those of generally tubular shape having a rounded, closed distal end and an open proximal end for receiving a residuum. Where a residuum includes an articulating joint (knee, elbow, etc.), the liner must handle compression and expansion of its portions that are near the point of articulation. Prosthetic suspension sleeves are configured to be provided over and around an articulating joint. Prosthetic suspension sleeves include those of generally tubular shape with opposing ends that are open.

Liners are generally of flexible, compressible materials. Materials available include silicone and other biocompatible or flexible polymers. A base material may include coatings to improve comfort. For example, a coating of Parylene (di-para-xylylene) may be provided.

Compression of these materials occurs on a side of the articulating joint that that is collapsed when the articulating joint transitions from an extended state to a flexed state. As this occurs, a fixed amount of material is forced into a tightening space. The material commonly buckles, bunches, and contorts to accommodate the forces acting upon it. Forces are transferred to the patient wearing the liner as the material takes on shapes that often cause the patient discomfort or even pain.

The inventors of the present disclosure have developed improved and enhanced liners and liner configurations and arrangements to alleviate issues relating to use of liners and experienced by prosthesis patients.

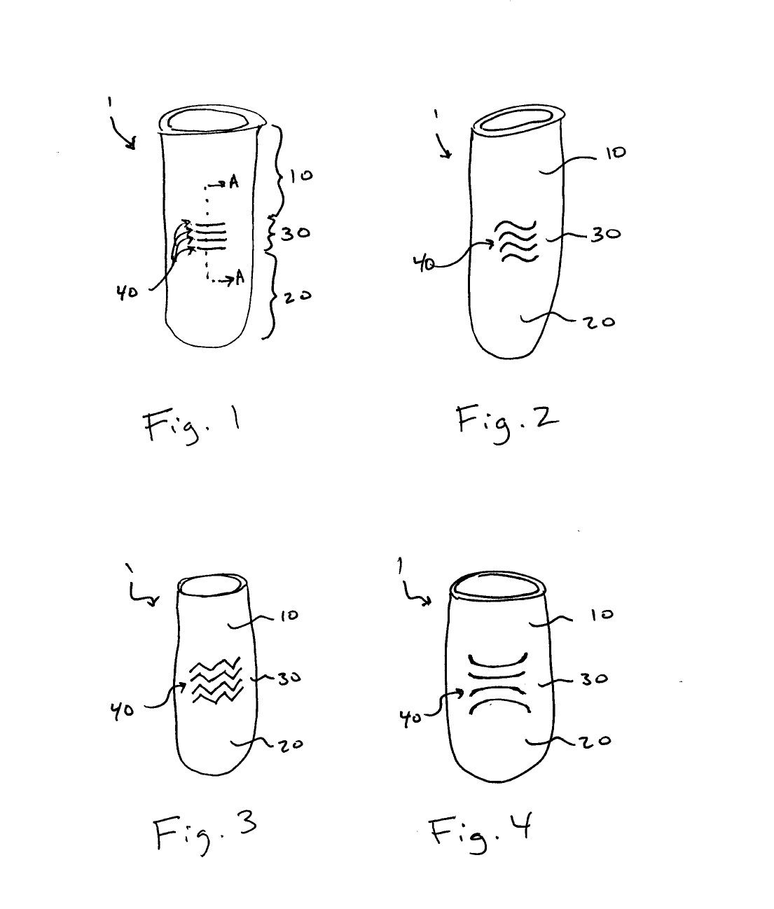

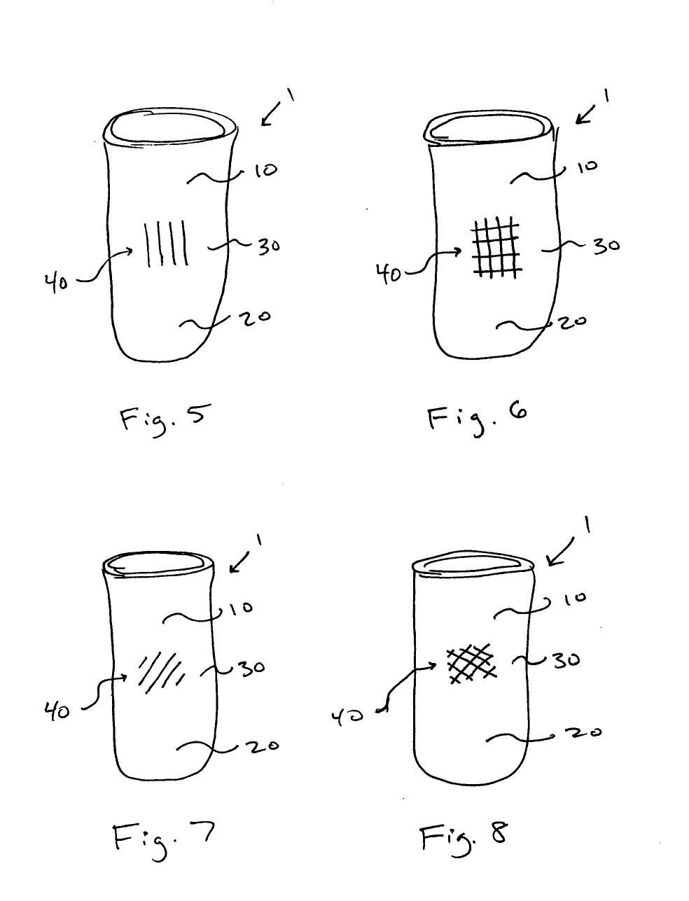

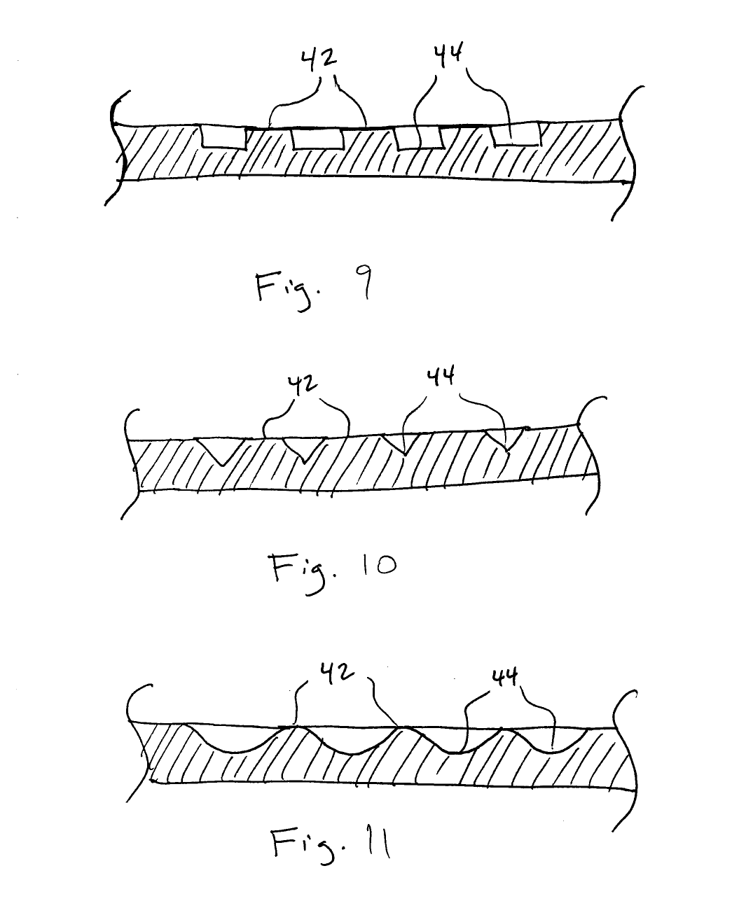

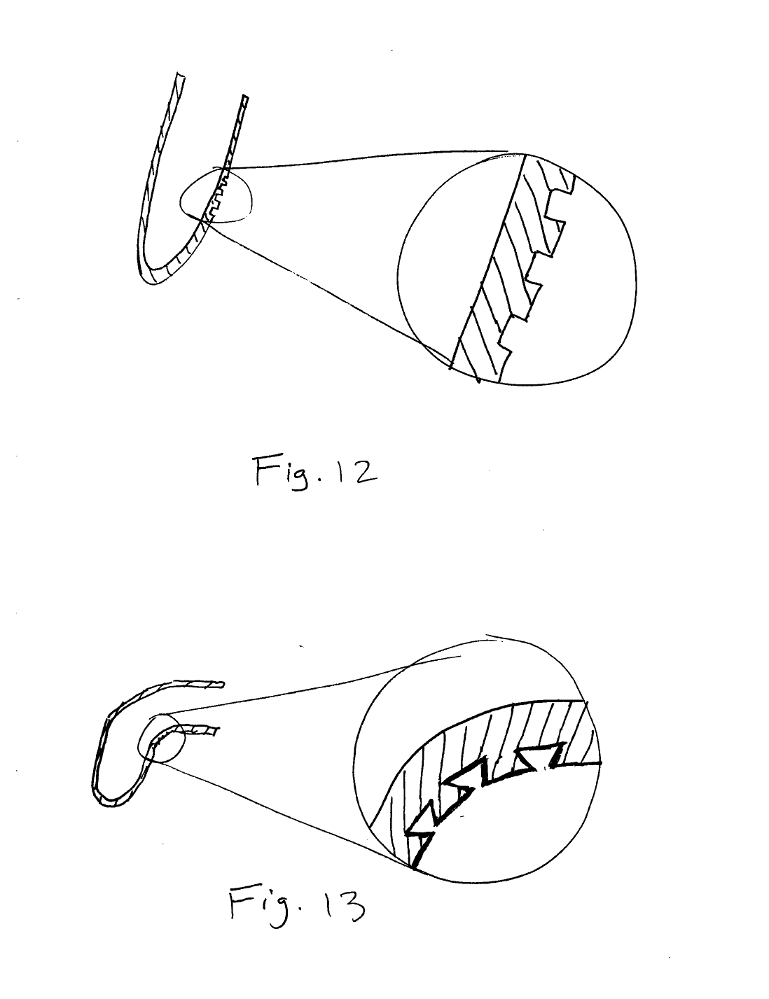

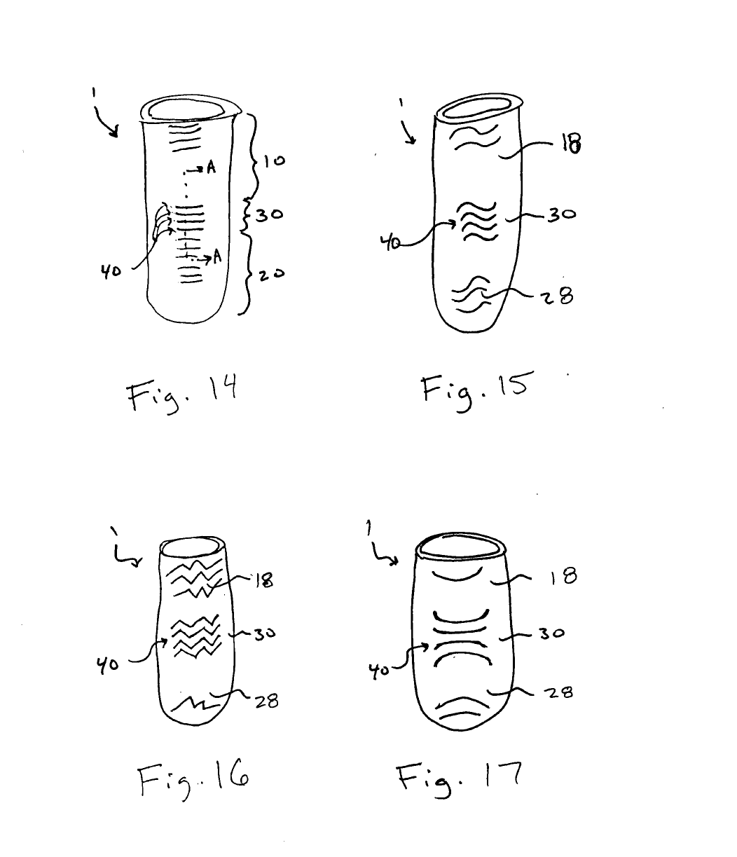

According to embodiments, as shown in FIGS. 1, 2, 3, and 4, liner 1 includes upper zone 10, lower zone 20, and medial zone 30 there between. Upper zone 10 is configured to align above an articulating joint of a body and cover a portion of a residuum above the articulating joint. Lower zone 20 is configured to align below an articulating joint of a body and cover a portion of a residuum below the articulating joint.

According to embodiments, medial zone 30 is configured to align along an articulating joint of a body and cover a the articulating joint. Medial zone 30 is a zone of flexure that improves flexing action of liner 1. Medial zone 30 includes ridges 40 aligned with a side of the articulating joint that is collapsed when the articulating joint transitions from an extended state to a flexed state. For example, medial zone 30 may be arranged to align with the popliteal fossa of a leg where a knee is the articulating joint. Medial zone 30 may be arranged to align with the cubital fossa of an arm where an elbow is the articulating joint. Such arrangements may be accomplished by considering the geometry of a residuum, including size, shape and length of portions above and below the articulating joint. Such considerations may be generalized or customized to individual needs.

According to embodiments, ridges 40 may form one or more of a variety of paths. As shown in FIG. 1, ridges 40 may form straight linear paths. As shown in FIG. 2, ridges 40 may form serpentine paths. As shown in FIG. 3, ridges 40 may form zig-zag paths. Any number of other paths and combinations thereof are contemplated by the present disclosure. According to embodiments, ridges 40, including peaks 42 and troughs 44, may include edges and transitions that are cornered, squared, scalloped, rounded, tapered, or combinations thereof.

According to embodiments, ridges 40 may align in parallel, diverge, or converge. For example, as shown in FIGS. 1,2, and 3, ridges 40 are parallel to each other as viewed along the longitudinal axis of liner 1. Such parallel alignment may provide a more uniform folding profile in a flexed state. As shown in FIG. 4, ridges 40 may approach more closely at a central portion and diverge at ends thereof. By further example, ridges 40 may diverge, converge, intersect, at one or more ends or central portions thereof. Such non-parallel alignment may provide programmable and variable folding profiles according to desired performance characteristics.

According to embodiments, as shown in FIG. 5, ridges 40 are aligned parallel to the longitudinal axis of liner 1. According to embodiments, as shown in FIGS. 6 and 8, ridges 40 may intersect at perpendicular or oblique angles to form crossing pathways. According to embodiments, as shown in FIG. 7, ridges 40 are aligned at an oblique angle relative to the longitudinal axis of liner 1. Ridges 40 provide variable thickness of liner or suspension sleeve material in medial zone 30. According to embodiments, ridges 40 include grooves, steps, ribbing, corrugation, waves, and other variable thickness configurations. As shown in FIGS. 9, 10 and 11, various shapes and geometries for ridges 40 may be provided. For example, ridges 40 may form peaks 42 and troughs 44. Each peak 42 and trough 44 may form one or more of a variety of shapes. For example, as shown in FIG. 9, peaks 42 and troughs 44 may form substantially flat surfaces with stepped transitions there between. By further example, as shown in FIG. 10, peaks 42 and troughs 44 may be pointed at ends, and have angled transitions there between. By further example, as shown in FIG. 11, peaks 42 and troughs 44 may form a sinusoidal wave or other smooth transitions. Any number of other geometries and combinations thereof are contemplated by the present disclosure. For example, geometric shapes could include one or more of an amorphous, sinusoidal, trapezoidal, triangular, rectangular, or squared shape.

According to embodiments, liner 1 may be of a single homogenous material or of multiple or heterogeneous materials. Liner 1, including ridges 40, may be formed by an injection molding process. Further, liner 1 may be formed by a multi-step process including formation of liner 1 without ridges and subsequently forming ridges 40 by removal or reformation of corresponding zones of liner 1. Liner 1 may include or be coupled with other components, such as interfacing materials to interact with residua or prosthesis devices.

According to embodiments, as shown in FIG. 12, ridges 40 of liner 1 in an extended state provide variable thickness as viewed in cross section. According to embodiments, as shown in FIG. 13, ridges 40 of liner 1 in a flexed state allow liner 1 to articulate without buckling or bunching. Bunching is avoided on outer surface where ridges 40 are present by reducing or removing the need for liner 1 to compress in order to facilitate articulation. Where ridges provide variable thickness of liner 1, the areas of relatively reduced thickness (i.e., troughs 44) allow the areas of relatively greater thickness (i.e., peaks 42) to approach each other. Troughs 44 allow the articulation to occur without substantially compressing the material. Thus, buckling that would otherwise occur in the presence of excessive material under excessive compression is avoided.

According to embodiments, other designs and configurations are contemplated by the present disclosure. As shown in FIG. 14, ridges 40 may extend into at least one of upper zone 10 and lower zone 20. As shown in FIGS. 15 and 16, upper ridges 18 and lower ridges 28 may be provided in upper and lower zones, respectively. As shown in FIG. 17, upper ridges 18 or lower ridges 28 may mimic or continue patterns provided in ridges 40 of medial zone 30.

While liners and suspension sleeves of various shapes and configurations are available including some having protrusions to enhance sealing-such solutions fail to resolve longstanding needs in the area of flexion about a joint and comfort of the patient. For example, such structures are generally located and configured to improve sealing at distal ends of a residuum, regardless of issues occurring at an articulating joint or even where no articulating joint is present. This offering does not address issues occurring at an articulating joint, particularly when the protrusions are not aligned with said articulating joint.

Further, according to embodiments of the present disclosure, ridges may be formed by troughs having lower thickness relative to other portions of the liner or suspension sleeve. For example, areas above or below a portion having ridges may have a substantially homogeneous or gradually tapered thickness and thereby provide a thickness greater than at the troughs of the ridges. Likewise, there may be provided smooth portions of the liner or suspension sleeve having substantially homogeneous thickness or gradually tapered thickness disposed across a central axis from the ridges. As such, the ridges may be targeted at a particular area where they may best address flexion of the articulating joint.

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all referenced listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “openend” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A process for improving fitting of a liner, comprising:
measuring a patient's residuum;
generating a molded liner; and
creating a zone of flexure in at least a portion of the molded liner.

2. The process of claim 1, wherein said zone of flexure further comprises a plurality of troughs aligned within predetermined zones.

3. The process of claim 2, wherein each of said troughs have a geometric shape, and the predetermined zone further comprises at least one of surface and density characteristics different from the balance of the liner.

4. A product by the process of claim 3.

5. A product, by the process of claim 3, said geometric shape being at least one selected from the group of squared, rectangular, triangular, trapezoidal, and sinusoidal.

6. A product by the process of claim 3, said trough being defined by an amorphous shape.

7. A liner, comprising, in combination:
an upper zone aligned above an articulating joint of a body;
a lower zone aligned below said articulating joint;
a medial zone comprising ridges aligned with a posterior portion of said articulating joint.

8. The liner of claim 7, wherein said ridges provide a profile to reduce bunching of said medial zone upon action of said articulating joint.

9. The liner of claim 7, wherein said ridges are substantially orthogonal to a longitudinal axis of said liner.

10. The liner of claim 7, wherein said ridges are substantially parallel.

11. The liner of claim 7, wherein said ridges are series of peaks and troughs.

12. The liner of claim 7, wherein liner has an extended state and a flexed state, wherein said medial zone does not bunch in said flexed state.

13. The liner of claim 7, wherein said posterior portion of said articulating joint is a popliteal fossa of a knee.

14. The liner of claim 7, wherein said posterior portion of said articulating joint is a cubital fossa of an elbow.

15. The liner of claim 7, being of a substantially homogenous material and formed by an injection molding process.

16. The liner of claim 11, said material being a silicone material at least partially coated with Parylene.

17. A liner, comprising, in combination:
a material of substantially homogenous thickness being tapered from an upper end to a lower end thereof;
ridges in a region corresponding to an articulating joint or a residuum, the ridges having troughs of thickness lower than that of surrounding portions of the liner; whereby the region corresponding to an articulating joint or residuum has at least one of a different durometer and altered surface characteristics.

18. A prosthetic suspension sleeve, comprising, in combination:
a tubular shape;
an upper zone of the tubular shape aligned above an articulating joint of a body;
a lower zone of the tubular shape aligned below said articulating joint;
a medial zone of the tubular shape comprising ridges aligned with an posterior portion of said articulating joint.

19. The prosthetic suspension sleeve of claim 18, wherein the ridges further comprises a plurality of troughs aligned within predetermined zones.

20. An enhanced liner system, which comprises, in combination:
at least a layer of material designed to buffer the interface with a prosthetic; and
at least one indented position positioned upon a predetermined location of said liner, enabling flexure of said layer to avoid bunching, pinching, or other insult to a user.
